(19) United States
(12) Patent Application Publication
(10) Pub. No.: US 2007/0049855 A1
(43) Pub. Date: Mar. 1, 2007

(54) ANKLE STIRRUP BRACE

(75) Inventor: Bradley D. MATTEAR, West Des Moines, IA (US)

Correspondence Address: ZARLEY LAW FIRM P.L.C., CAPITAL SQUARE, 400 LOCUST, SUITE 200, DES MOINES, IA, 50309-2350, US

(73) Assignee: RESTORATIVE CARE OF AMERICA INCORPORATED, St. Petersburg, FL (US)

(21) Appl. No.: 11/162,012

(22) Filed: Aug. 25, 2005

(51) Int. Cl. A61F 5/00

(52) U.S. Cl. 602/27

(57) ABSTRACT

An ankle brace having a pair of shells with a pad attached to the inner surface of the shells. Attached to the pads is a U-shaped strip positioned to cup the medial and lateral malleous portions of an ankle and extending from the tip of the shell to the opposite end of the shell. At least one strap is connected to the shells and extends around the shell, and an ankle strap is adjustably connected to the shells.

### BACKGROUND OF THE INVENTION

This invention is directed to an ankle brace and more specifically, an ankle brace designed to maximize edema release.

Ankle braces are well-known in the art. Generally, these braces are designed for application in the orthopedic medical field, where ankle sprains or factures are often supported and immobilized with a splint or brace so that a patient can continue to walk while the injury heals. Some ankle injuries result in edema, or swelling around the ankle and forefoot.

Current ankle braces do not effectively maximize edema release, provide circumferential support, or are sufficiently adjustable to provide for a full range of motion for both planter and dorsi-flexion. Accordingly, there is a need for an ankle brace that addresses these problems.

Therefore, an objective of this invention is to provide an ankle brace that maximizes edema release.

Another objective of the invention is to provide an ankle brace that is easily adjustable and allows for a full range of motion.

A still further objective is to provide an ankle brace that provides greater support around a patient's leg.

These and other objectives will be apparent to those skilled in the art based on the following written description.

### SUMMARY OF THE INVENTION

An ankle brace having a pair of shells with a pad attached to the inner surface of the shells. Attached to the pads is a U-shaped strip positioned to cup the medial and lateral malleous portions of an ankle and extending from the tip of the shell to the opposite end of the shell. At least one strap is connected to the shells and extends around the shell, and an ankle strap is adjustably connected to the shells.

### BRIEF DESCRIPTION OF THE DRAWINGS

FIG. 1 is a perspective front view of an ankle brace; and

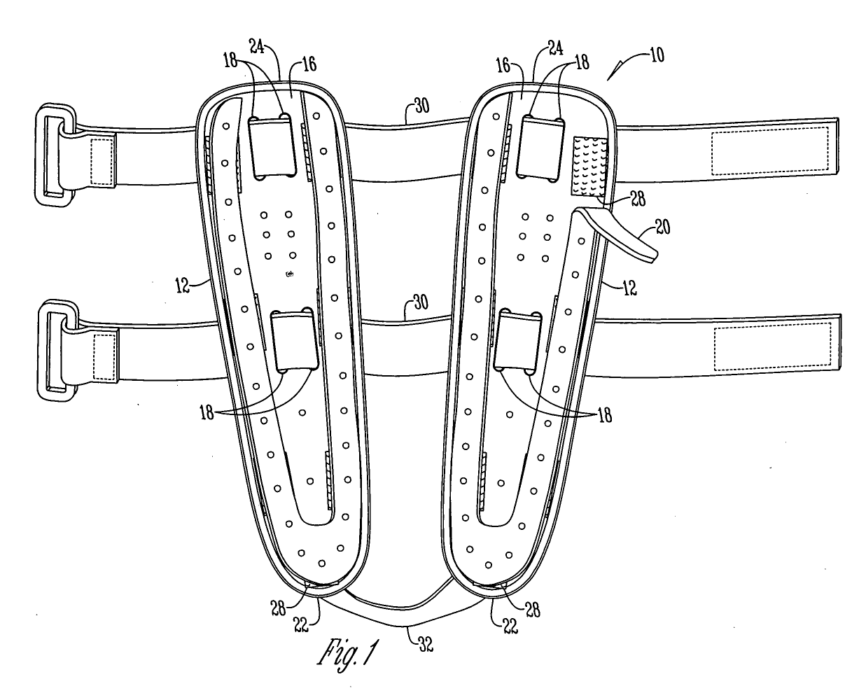

FIG. 2 is a perspective back view of an ankle brace; and

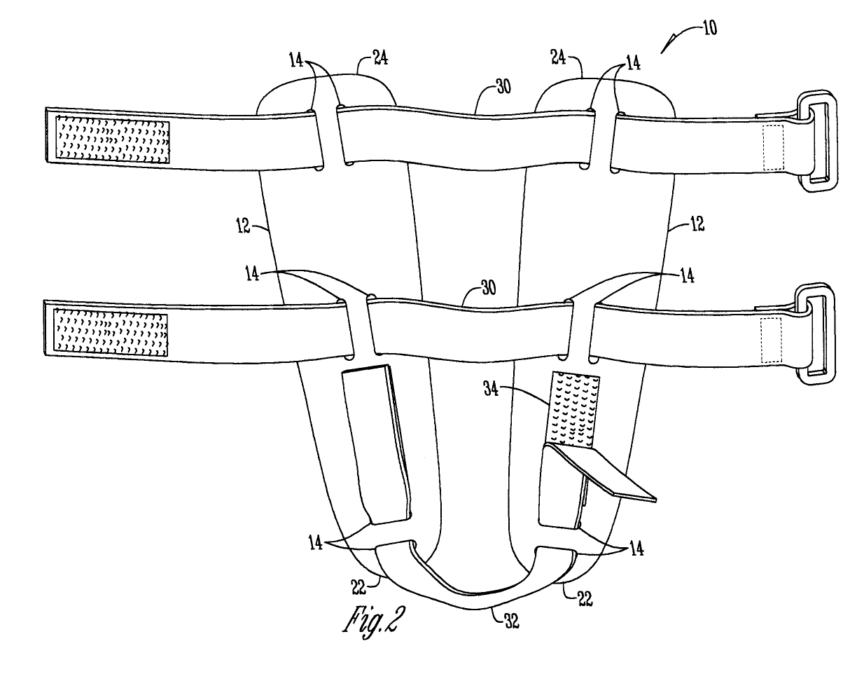

FIG. 3 is a front end view of an ankle brace fitted to a patient's leg.

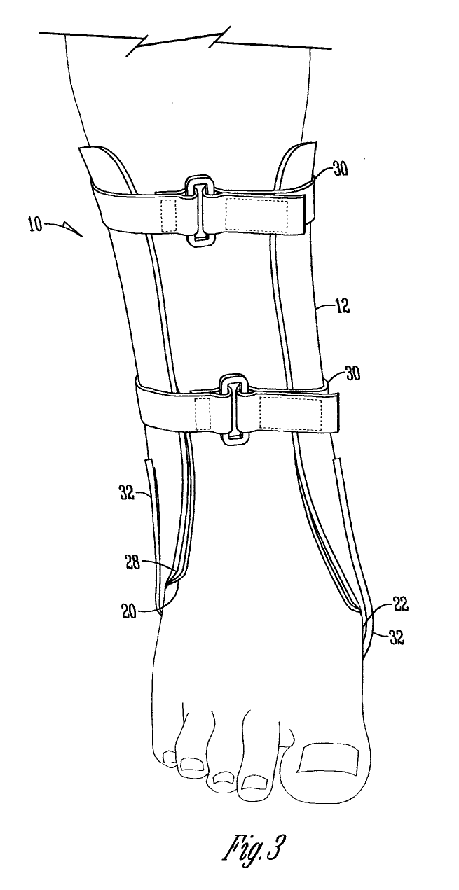

### DETAILED DESCRIPTION OF THE PREFERRED EMBODIMENT

Referring to the Figures, an ankle brace 10 has a pair of shells 12 formed to fit the medial and lateral malleous portions of an individual's leg. The shells 12 are preferably made of either poly-propropulene or poly-ethylene or similar thermoplastic to provide either a firm or flexible fit as desired.

The shells 12 have a plurality of slots 14 that extend through the shells 12. Attached to the inner surface of the shells 12 are pads 16. The pads 16 have a plurality of openings 18 that are aligned with the slots 14 in the shells 12. Removably attached to the outer perimeter of the pads 16 are U-shaped/horseshoe strips 20 that are positioned to cup the medial and lateral malleous portions of an ankle. The strips 20 extend from the tip 22 of the shell 12 and pad 16 to the opposite end 24 and are preferably perforated and made of neoprene or closed cell foam. The strip 20 is positioned to aid in edema release and by extending the strip 20 from end 24 to the tip 22 edema release is maximized. The strips 20 are removably attached to the pad 16 in any conventional manner such as with a Velcro® surface 26 that mates with Velcro® patches 28 that are glued to the pad 16.

A pair of hook and loop straps 30 extend through slots 14 and 18, around the outer surface of the shells 12 where the straps 30 are secured and tightened to provide firm circumferential support about a patient's leg.

Extending through a slot 14 and 18 adjacent the tip 22 is an ankle strap 32. To aid with easy adjustment the strap 32 has a Velcro® surface that mates with Velcro® strips 34 attached to the outer surface of the shells 12.

The brace 10 is attached to one's ankle by threading straps 30 and 32 through slots 14 ands 18. Then shells 12 are placed on one's ankle and lower leg such that strips 20 cup the medial and lateral malleous portions of the ankle. Strap 32 is then adjusted through a connection with strip 34 for a desired fit which is either stable for post fracture conditions or allowing for a full range of motion for either walking, running, or other athletic competition. Finally, the ends of straps 30 are secured and tightened to provide support about the patient's leg.

The brace described is capable of providing support for medial/lateral acute ankle sprain or strain, and anterior talofubular ligament and major deltoid ligament damage (Grade 1-3). In addition, the brace provides support for chronic ankle instabilities, prophylactic use, and is helpful as a post surgical device to aid in rehab and provide protection for bony structures due to contusions. The ankle brace described, at the least, meets all the stated objectives.

What is claimed is:

1. An ankle stirrup brace, comprising:
a pair of shells having pads on the inner surface of the shells;
the shells having at least one slot extending through the shells;
a pair of U-shaped strips attached to an outer periphery of the pads and extending from a tip of the shell to an opposite end of the shell;
at least one strap that is attached to the shells and extends around the shells; and
an adjustable ankle strap attached to the shells.

2. The brace of claim 1 wherein the strips are perforated and made of closed cell foam.

3. The brace of claim 1 wherein the strips are removably attached to the pads.

4. The brace of claim 1 wherein the pads have at least one opening that is aligned with the slot in the shell.

5. The brace of claim 1 wherein the strap extends through the slot in the shell.

6. The brace of claim 1 wherein the strap extends through the opening in the pads.

7. The brace of claim 1 wherein the strap has a Velcro® surface that mates with a Velcro® strip attached to an outer surface of the shells.

8. The brace of claim 1 wherein an ankle strap extends from a tip of one shell to a tip of the other shell.

9. The ankle strap of claim 8 wherein the ankle strap extends through a slot adjacent the tip of the shell.

10. The ankle strap of claim 8 wherein the ankle strap has a Velcro® surface that mates with a Velcro® strip attached to an outer surface of the shells.

11. The brace of claim 1 wherein the strap has a Velcro® surface that mates with a Velcro® surface of the strap.
